(19) United States
(12) Patent Application Publication
(10) Pub. No.: US 2006/0159725 A1
(43) Pub. Date: Jul. 20, 2006

(54) Herbal compositions

(76) Inventors: Shiu Fun Pang, Kowloon (HK); Sharon Luk, Sunshine City (HK); Edgar Shiu Lam Liu, New Teritories (HK); Hongtao Xing, New Territories (HK)

Correspondence Address: FISH & RICHARDSON PC, P.O. BOX 1022, MINNEAPOLIS, MN, 55440-1022, US

(21) Appl. No.: 11/292,294

(22) Filed: Dec. 1, 2005

Related U.S. Application Data
(60) Provisional application No. 60/632,402, filed on Dec. 1, 2004.

(51) Int. Cl. A61K 47/00; A61K 36/06; A61K 36/254; A61K 36/906; A61K 36/48; A61K 36/28; A61K 36/73; A61K 36/258

(52) U.S. Cl. 424/439; 424/728; 424/195.16; 424/756; 424/764; 424/757; 424/765

(57) ABSTRACT

A composition containing extracts obtained form Fructus Crataegi, Semen Zizyphus, Fructus Schizandrae, and Panax Ginseng. Also disclosed is a method of treating cancer or modulating an immune response in a subject and a method of inducing a cytokine in a cell with the composition.

### CROSS REFERENCE TO RELATED APPLICATION

Pursuant to 35 U.S.C. §119(e), this application claims priority to U.S. Provisional Application Ser. No. 60/632,402, filed Dec. 1, 2004, the contents of which are hereby incorporated by reference.

### BACKGROUND

Immune responses involve cascades of well-coordinated cellular and molecular events in response to various intrinsic and extrinsic stimuli. Cytokines, secreted by cells (particularly, those of the haematopoietic origin), play important roles in mediating various immune responses. Indeed, the pattern of cytokine production characterizes the type of immune responses. For example, a typical Th1 response, or cytotoxic T lymphocyte response, is characterized by an up-regulation of three cytokines, i.e., interleukin-2 (IL-2), interferon-gamma (IFN-γ) and tumor necrosis factor-beta (TNF-β). A typical Th2 response, or memory response, is characterized by an up-regulation of another three cytokines, i.e., interleukin-4 (IL-4), interleukin-6 (IL-6), and interleukin-10 (IL-10). Upon activation, cytokine release is usually a short-term event that effectuates within hours, followed by cellular responses hours or days later. Induction of certain cytokines can be effective in treating various diseases, such as cancer.

Natural botanical products have a long history in medical applications. Many herbs have been found to possess immunomodulating activities. Further, natural botanical products are generally safe and have little side effects. There remains a need to develop drugs based on natural botanical products.

### SUMMARY

In one aspect, this invention features a composition containing extracts obtained from at least two of the following herbs: Fructus Crataegi (i.e., hawthorn), Semen Zizyphus (i.e., jujube), Fructus Schizandrae (i.e., “Wu Wei Zi” in Chinese), and Panax Ginseng (i.e., ginseng). It can be in a dry form (e.g., powder) or in an aqueous form (e.g., a water solution). The composition may be made by obtaining extracts from the ingredient herbs separately and then mixing them together, or by mixing the ingredient herbs together first and then obtaining the extracts from the herbal mixture.

The composition of the invention can be a pharmaceutical formulation, e.g., a tablet, a capsule, powder, a dispersion, a solution, or a gel. It can also be a food product or dietary supplement. Examples include tea (including tea drink and the contents of a tea bag), soft drink, juice, milk, coffee, soup, beer, chewing gum, seasonings, cookies, cereals, and chocolates.

In another aspect, this invention features a method of treating cancers (including liver, gastric, prostate, and breast cancers) and a method of modulating an immune response, such as by inducing of tumor necrosis factor-alpha (TNF-α) or IL-6. The method includes administering to a subject in need of cancer treatment an effective amount of a composition of the invention.

In still another aspect, this invention features a method of modulating an immune response by inducing secretion of cytokines, such as tumor necrosis factor-alpha (TNF-α) or IL-6, from immune cells. The method includes contacting the cell, which can be present in a cell culture, a tissue, or a subject, with an effective amount of a composition of the present invention. An example of such a cell is a lymphocyte, such as a peripheral blood mononuclear cell, a macrophage, or a T lymphocyte.

Also within the scope of this invention is the composition described above for use in treating cancer, modulating immune response, and inducing cytokines, and the use of such a composition for the manufacture of a medicament for the just-mentioned purposes.

The details of one or more embodiments of the invention are set forth in the description below. Other features, objects, and advantages of the invention will be apparent from the description and from the claims.

### DETAILED DESCRIPTION

This invention provides a method of treating cancer using a composition that contains at least two of the following four herbs, i.e., a Fructus Crataegi extract, a Semen Zizyphus extract, a Fructus Schizandrae extract, and a Panax Ginseng extract. Exemplary quantities of the ingredients of this composition are: Fructus Crataegi: 3-30 g (e.g., 13 g); Semen Zizyphus: 3-30 g (e.g., 13 g); Fructus Schizandrae: 4-20 g (e.g., 8 g); Panax ginseng: 1-15 g (e.g., 4 g ), or in quantities of the same relative ratio to those listed above.

The extracts of Fructus Crataegi, Semen Zizyphus, Fructus Schizandrae can be obtained from the fruits of hawthorn, jujube, and Wu Wei Zi, repsectively, and the Panax Ginseng extract can be obtained from ginseng roots. These herbs are commercially available. After authenticating each individual herb to be used, conventional methods may be used to process the composition of the present invention into a form suitable for administering to human subjects. Those conventional methods are either described in pertinent literature or commonly used by practitioners of herbal medicine. By way of non-limiting example, a suitable form can be tinctures, decoctions, or dry extracts. Extracts may be further processed into pills, tablets, capsules or injections.

A tincture is prepared by suspending herbs in a solution of alcohol, such as wine or liquor. After a period time of suspension, the liquid (the alcohol solution) may be administered two or three times a day, one teaspoon each time. A decoction is the most common form of herbal preparations. It is traditionally prepared in a clay pot, but nowadays it can also be prepared in glass, enamel or stainless steel containers. The herbs should be soaked for a period of time in a proper amount of water and then quickly brought to a boil and simmered until the amount of water is reduced by half.

An extract is a concentrated preparation of the essential constituents of the medicinal herb. Typically, the essential constituents are extracted from the herbs by suspending the herbs in an appropriate choice of solvent, typically, water, ethanol/water mixture, methanol, butanol, iso-butanol, acetone, hexane, petroleum ether or other suitable organic solvents. The extracting process may be further facilitated by means of maceration, percolation, repercolation, counter-current extraction, turbo-extraction, or by carbon-dioxide hypercritical (temperature/pressure) extraction. After filtration to rid of herb debris, the extracting solution may be further evaporated and thus concentrated to yield a soft extract (extractum spissum) or eventually a dried extract (extracum siccum), by means of spray drying, vacuum oven drying, fluid-bed drying, or freeze-drying. The soft extract or dried extract may be further dissolved in a suitable liquid to a desired concentration for administering or processed into a form such as pills, capsules, and injections.

A particular embodiment of the present invention, referred to as CKBM, is a composition comprising Fructus Crataegi: 13 g; Semen Zizyphus: 13 g; Fructus Schizandrae: 8 g; and Panax ginseng: 4 g; or in quantities of the same relative ratio. The weights specified above represent the relative weights of the ingredient herbs prior to the extracting process. The ingredient herbs of CKBM may be mixed before being extracted and subjected to the extracting process together. Alternatively, the herbs may be extracted individually and then the resulting extracts are mixed. CKBM may be in a solid form (powder, capsule, tablet, etc) or in a solution form.

In another aspect of the invention, the composition of present invention may further comprise one or more of the following ingredients: an extract obtained from yeast (e.g., Saccharomyces cerevisiae), soybean, green bean, apple, or honey. The extract of yeast may be prepared by a procedure known in the art. The composition may be sweetened, if necessary, by adding a sweetener such as sorbitol, maltitol, hydrogenated glucose syrup, hydrogenated starch hydrolyzate, high fructose corn syrup, cane sugar, beet sugar, pectin, and sucralose.

An example of the above-described composition is a powder, which can be conveniently used to prepare an aqueous solution, such as beverages (e.g., tea or juice). The composition can also be a dietary supplement or a food product. When the composition is used as a dietary supplement, it is preferred that additional nutrients, such as minerals or amino acids, be included.

Also within the scope of this invention is a pharmaceutical composition that contains an effective amount of a suitable composition among those described above and a pharmaceutically acceptable carrier. The carrier in the pharmaceutical composition must be “acceptable” in the sense that it is compatible with the active ingredients of the composition (and preferably, capable of stabilizing the active ingredients) and not deleterious to the subject to be treated. The pharmaceutical composition can be in the form of a tablet, a capsule, powder, dispersion, a solution, or a gel. Lactose and corn starch are commonly used as carriers for capsules and tablets. Lubricating agents, such as magnesium stearate, are typically added to form tablets. Examples of other carriers include colloidal silicon oxide, cellulose, sodium lauryl sulfate, and D&C Yellow #10.

Further, this invention covers a method of treating cancer by administering (e.g., orally) an effective amount of a suitable composition among those described above to a subject. The term “treating” refers to the administration of an effective amount of the composition to a subject, who suffers from one or more of the just-mentioned diseases, or a symptom or a predisposition of one of more of them, with the purpose to cure, alleviate, relieve, remedy, or ameliorate one or more of the diseases, or the symptoms or the predispositions of one or more of them.

Effective doses will vary, as recognized by those skilled in the art, depending on the types of diseases to be treated, route of administration, excipient usage, and the possibility of co-usage with other therapeutic treatment. Exemplary dosages of the liquid form of the composition described above range from 45-540 ml per day. Exemplary dosages of the dry form of the composition described above range from 50-5,000 mg per day.

This invention also covers a method of modulating an immune response in a subject by administering an effective amount of the composition described above to the subject. Further, this invention covers a method of inducing a cytokine (e.g., IL-6 or TNF-α) in a cell by contacting the cell with an effective amount of the composition described above.

The compositions described above can be preliminarily screened for their efficacy in treating cancer, modulating an immune response, or inducing a cytokine by in vitro assays (see Examples 2-4 below) and then confirmed by animal experiments (see Examples 5-8 below) and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLE 1

### Preparation of CKBM

A CKBM solution was prepared as follows: (1) mixing Fructus Crataegi (13 g), Semen Zizyphus (13 g), Fructus Schizandrae (8 g), and Panax ginseng (4 g); (2) first extraction with 448.3 ml H₂O at 100° C. for 100 minutes; (3) second extraction with 336.24 ml H₂O at 100° C. for 60 minutes; (4) third extraction with 224.16 ml H₂O at 100° C. for 40 minutes; and (5) combining the three extracts and adding water to reach a final volume of about 1 liter.

### EXAMPLE 2

### Induction of Cytokine in Peripheral Blood Mononuclear Cells by CKBM

Ninty milliliters of the CKBM solution prepared from Example 1 was centrifuged for 30 minutes. The supernatant was then filtered using a 0.22 mm filter. The filtered CKBM was stored at −20° C. Prior to use, CKBM was thawed and kept at 4° C. It was then serially diluted in complete RPMI 1640 culture medium with 10% heat-inactivated fetal bovine serum (v/v), 1% penicillin-streptomysin (w/v), and 2 g/L sodium hydrogen carbonate. The tested CKBM concentrations were 10, 5, 2.5, 1, and 0.5% (v/v).

Blood samples were obtained from healthy human subjects. Peripheral blood mononuclear cells (PBMCs) from these blood samples were prepared following routine procedures known in the art. PBMCs were then seeded at 1×10⁵ cells per well in a 24 well plate. Five hundred microliters of CKBM at various concentrations (0, 0.5, 1.0, 2.5, 5, and 10% in RPMI (v/v)) were added to appropriate cells, which were subsequently incubated for 4, 24, and 48 hours. Positive controls, including phytohemagglutinin at 1 or 5 μg/ml, phorbol myristate acetate at 0.4 μg/ml, and lonoycin 0.4 μg/ml, were added to appropriate cells, which were also incubated for the aforementioned time periods. The culture medium from each well was then collected at the end of each incubation period and the concentrations of cytokines were measured using cytometric bead array (CBA) kits. Raw data of cytokine induction was further analyzed using standard statistical assays. For example, ANOVA assay was used to reveal the difference in cytokine or its receptor levels among various CKBM dosage groups and time points. Bonferroni's or Dunnett's test was used whenever necessary as a post-hoc analysis. Statistical significance was set at 0.05 and SPSS 10.0 was used to execute all data analysis.

The results show that CKBM induced the secretion of both TNF-α and IL-6, but not IL-2, IL-4, IL-10 and IFN-γ, in PBMCs. The induction of TNF-α reached its peak at 4 hours after CKBM treatment, sustained to 24 hours and then declined at 48 hours. CKBM at concentrations of 0.5, 1.0, and 2.5% significantly increased TNF-α secretion and a peak response was noted at 1.0%. The peak level of TNF-α reached about 101.4 pg/ml, which was about 36 fold of the minimally detectable limit of this assay. The TNF-α production decreased when 5 and 10% of CKBM were used.

The induction of IL-6 had a different pattern compared to that of TNF-α. Little induction was noticed four hours after treatment at any CKBM concentration, but a significant increase was observed at 24 and 48 hours after CKBM treatment. The peak median response was found at 24 hours after treatment of 2.5% CKBM. The peak level of IL-6 reached about 770.7 pg/ml, which was about 257 fold of the lowest detection limit of this assay.

The levels of IL-6 and TNF-α were further examined using standard ELISA kits and the results were consistent with those obtained by CBA kits even though there were individual variations in the magnitude of responses.

In addition, standard flow cytometry analysis was performed to detect intracellular TNF-α and IL-6. In particular, PBMCs at 1×10⁵ cells in 0.5 ml RPMI were added to a polypropylene round-bottom tube and were incubated with CKBM at various concentrations or with positive controls for 16 hours at 37° C. Brefeldin A (BFA) at a concentration of 10 μg/ml was added to the mixture for the last 4 hours of the incubation period. After a brief wash with phosphate buffered saline (PBS), PBMCs were lysed by FACS lysing solution for 10 minutes at room temperature. PBMCs were then washed once with PBS containing 0.5% BSA and were then permeabilized by FACS permeabilizing solution for 10 minutes in dark. After a brief wash, PBMCs were incubated with 10 μl of anti-TNF-α or anti-IL-6 monoclonal antibodies and isotype controls for 30 minutes. After another wash, PBMCs were fixed with 1% formaldehyde before being subjected to flow cytometry analysis. The results show that intracellular TNF-α did not show a significant increase after CKBM treatment for 16 hours. Intracellular IL-6, to the contrary, showed a small but significant increase 16 hours after CKBM treatment. The response was dose-dependent and peaked at a concentration between 1% and 2.5%.

Next, a TNF-α receptor, p75, was examined by standard ELISA assay after PBMCs were incubated with various doses of CKBM for 4, 24 and 48 hours. The level of p75 did not change at the 4-hour time-point, but started increasing at 24 hours and peaked at 48 hours after CKBM treatment. ANOVA and Bonferroni post-hoc test showed that all results were statistically significant.

Finally, the cytotoxicity of CKBM on PBMC was examined by the standard MTT (3-[4,5-dimethylthizol-2-yl]-2,5-diphenyl tetrazolium bromide) labeled cell cytotoxicity assay. It is well-known that the MTT assay is a quantitative calorimetric metabolic assay for the determination of cell survival and proliferation. Dunnett's test (>control) showed that CKBM at the concentrations of 0.5, 1.0, and 2.5% had no cytotoxicity effect on PBMCs, while CKBM at the concentration of 5% and above showed a significant increase in cytotoxicity compared to the control (p<0.05).

### EXAMPLE 3

### Modulation of Cellular Immune Response in Blood from HIV-Positive and HIV-Negative Individuals by CKBM-A01

A CKBM-A01 solution was prepared in a manner identical to the CKBM solution prepared from Example 1 except that it contains yeast extract (≦0.1% by weight). The effects of CKBM-A01 in modulating cellular immune response were investigated in blood from HIV-positive and HIV-negative individuals. In this investigation, two studies were performed: (1) quantification of intracellular cytokine staining (ICS); and (2) identification of the cell population secreting the TFN-α.

Each study used the blood samples collected from the patients selected as follows. Five out of ten HIV-1-infected patients (chronically infected, retroviral drugs naive, and symptomatic) and five out of ten HIV-1-negative healthy controls were selected for blood collection (60 ml of ACD peripheral blood) by the AIDS Research Center in MGH. All individuals were treatment naive. CD4+ T cell counts, HIV-1-viral load and HLA class I types were determined on each patient. Table 1 shows the blood data of the first group of patients selected for the first study.

TABLE 1

| Patient | HIV | CD4 | Viral | |
|---|---|---|---|---|
| # | status | counts | load | HLA type |
| N1 | Neg | >600 | — | A24/26, B40/—, Cw02/15 |
| N2 | Neg | >600 | — | A01/29, B07/51, Cw04/07 |
| N3 | Neg | >600 | — | A02/—, B15/44, Cw03/05 |
| N4 | Neg | >600 | — | A01/03, B15/57, Cw0l/06 |
| N5 | Neg | >600 | — | A02/26, B38/39, Cwl2/— |
| P1 | Pos | 602 | 1210 | A02/—, B14/40, Cw03/08 |
| P2 | Pos | 637 | 5000 | A02/11, B15/40, Cw04/07 |
| P3 | Pos | 1080 | <50 | A03/26, B44/51, Cw05/15 |
| P4 | Pos | 891 | 590 | A01/30, B27/57, Cw0l/06 |
| P5 | Pos | 335 | 58980 | A03/24, B07/15, Cw0l/07 |

The potential toxicity of CKBM-A01 was tested on the blood samples, using concentrations in the range of 0.25%-2%. Table 2 shows that short incubation (<24 hrs) with CKBM-A01 did not reduce the viability of blood cells, while longer incubation (72 hrs) did, in particular at the high concentration.

TABLE 2

| | Control | | | | |
|---|---|---|---|---|---|
| Viability | (Million/ml) | 2% | 1% | 0.5% | 0.25% |
| 24 hrs | 0.76 | 0.75 | 0.88 | 0.91 | 0.76 |
| 72 hrs | 0.46 | 0.29 | 0.31 | 0.31 | 0.33 |

Quantification of intracellular cytokine staining (ICS) was performed on blood samples by flow cytometry. The results show that for interferon-gamma ICS (IFN-γ ICS), no enhancement of antigen-specific IFN-γ production of CD4+ T cells, CD8+ T cells or the CD3−/CD4−/CD8− population was observed in the presence of CKBM-A01 for both HIV-negative and HIV-positive individuals. The data for TFN-α ICS demonstrate an enhancement of TFN-α production in the presence of CKBM-A01 in some individuals (1 out of 3 HIV-negative and 3 out of 4 HIV-positive patients to date).

Table 3 shows the blood data of the second group of patients selected for the second study, i.e., identification of the cell secreting TFN-α.

TABLE 3

| Patient | HIV | CD4 | Viral |
|---|---|---|---|
| # | status | counts | load |
| N6 | Neg | >600 | — |
| N7 | Neg | >600 | — |
| N8 | Neg | >600 | — |
| N9 | Neg | >600 | — |
| Nl0 | Neg | >600 | — |
| P6 | Pos | 354 | 3390 |
| P7 | Pos | 617 | <50 |
| P8 | Pos | 545 | <50 |
| P9 | Pos | 568 | 5800 |
| Pl0 | Pos | 315 | 3634 |

To determine the cell population responding to CKBM-A01 to secret TFN-α, the intracellular TFN-α secretion with and without the presence of 1% CKBM-A 01 was assessed by co-staining the freshly isolated peripheral blood mononuclear cells with a variety of different surface-staining antibodies, such as CD3, CD4, CD8, CD11b, CD14 (/CD33), CD56 and CD19.

Table 4 shows the experimental results from the blood of the above five HIV-1 negative and five HIV-1-infected individuals. Cells from all 10 subjects responded to CKBM-A01 with secretion of TFN-α The cell subsets of CD3+ CD8+ and CD3+ CD4+ T lymphocytes did not respond to CKBM-A01, while the subsets of CD11b+ (mainly neutrophils) and CD14+ (CD33+) (mainly monocytes or macrophages) cells were the major cell subsets secreting TFN-α in response to CKBM-A01. A small proportion of CD3-CD56+ cells (NK cells) did also respond to CKBM-A01; however, it differed in HIV-1 negative and HIV-1-infected subjects: for HIV-1-infected individuals (i.e., P6-P10), no significant difference in TFN-α secretion by CD3-CD56+ cells was observed with or without the presence of CKBM-A01 (p=0.42); and for HIV-1 negative individual (i.e, N6-N10), this difference was significant (p=0.025). This could indicate that the CD3-CD56+ cells may lose their capacity to respond to CKBM-A01 after HIV-1 infection in some infected individuals.

TABLE 4

| | CD3+CD8+ | | CD3+CD4+ | | CD11b+ | | CD14+ | | CD3−CD56+* | |
|---|---|---|---|---|---|---|---|---|---|---|
| | no | 1% | no | 1% | no | 1% | no | 1% | no | 1% |
| | drug | drug | drug | drug | drug | drug | drug | drug | drug | drug |
| P6 | 0.2 | .36 | .04 | .01 | | | .83 | 38.64 | .4 | .17 |
| P7 | .01 | .05 | .09 | .06 | .24 | 4.59 | .27 | 58.66 | .34 | 1.94 |
| P8 | .049 | .07 | .14 | .021 | .63 | 8.88 | .77 | 24.6 | .27 | .32 |
| P9 | .029 | .03 | .025 | .08 | .31 | 9.17 | .61 | 14.4 | .23 | .33 |
| Pl0 | .043 | .053 | .22 | .021 | .073 | 2.54 | .41 | 17 | .13 | .14 |
| N6 | .03 | .02 | .03 | .04 | 1.51 | 29.38 | .35 | 14.19 | .39 | 1.41 |
| N7 | .09 | .01 | .05 | .12 | .51 | 9.45 | .15 | 5.45 | .23 | .46 |
| N8 | .02 | .05 | .07 | .12 | 1.52 | 11.04 | .62 | 4.04 | .06 | 1.04 |
| N9 | .03 | .01 | .04 | .03 | .62 | 15.72 | 1.35 | 7.43 | .16 | 1.05 |
| N10 | .04 | .06 | .23 | .01 | .36 | 14.08 | 1.48 | 8.48 | .24 | .4 |
| p value | .6508 | | .1513 | | .0029 | | .0078 | | .0339 | |

Thus, TFN-α production was enhanced in the presence of CKBM-A01 in the cell subsets in both HIV-1 positive and HIV-1 negative individuals. The cell subsets secreting TFN-α after being stimulated with CKBM-A01 were mainly monocytes (macrophage) and neutrophiles, and NK cells to a less extent, as determined by surface staining with CD11b, CD14 (/CD33), and CD56 markers of these TFN-α positive populations. The antigen-specific IFN-γ production in HIV-1 positive and HIV-1 negative individuals was not enhanced in the presence of CKBM-A01.

### EXAMPLE 4

### Inhibition of the Growth of Human Hepatocellular Carcinoma HepG2 Cells by CKBM

The CKBM solution prepared from Example 1 was filtered by a 0.22 μm syringe filter and diluted to appropriate concentrations using a culture medium (see Example 2).

Human hepatoma HepG2 cell line (ATCC number HB-8065), normal liver cell line WRL-68 (ATCC number CL-48), and normal skin fibroblasts cell line Hs68 (ATCC number CRL-1635) were purchased from the American Type Culture Collection. All cells were cultured following the instructions in ATCC's manual.

The cytotoxicity effect of CKBM on the above cell lines were first examined using the standard MTT assay. The results show that the HepG2 cell survival rate was about 50% when cells were treated with 10% CKBM for 48 hours. No significant inhibition of cell growth, however, was observed in Hs68 and WRL-68 cells treated with 10% CKBM for the same time period. Even when treated with 14% CKBM, which resulted in about 70% growth inhibition of the HepG2 cells, more than 80% of the Hs68 and WRL-68 cells still survived. These results show that CKBM has a specific anti-proliferation effect on hepatoma cells.

Next, DNA fragmentation was examined on HepG2 cells treated with CKBM to determine whether CKBM could induce cell apoptosis. DNA samples from HepG2 cell lines were prepared according to conventional methods and subjected to electrophoresis at 80 v on a 1.5% agrose gel (w/v). DNA fragmentation was observed in HepG2 cells incubated with 14% CKBM for 48 hours, but not in un-treated HepG2 cells, indicating that CKBM induced cell apoptosis. Further, standard western-blot assays showed an increased level of p53 (an activator of apoptosis), and a decreased level of Bcl-2 (a suppressor of apoptosis), after HepG2 cells were incubated with 12% and 14% CKBM for 48 hours. The level of caspase 3, one of the most crucial caspases in the apoptotic cascade, was also down-regulated by the treatment of CKBM. These results indicate that CKBM inhibited hepatoma HepG2 cell growth by inducing cell apoptosis.

### EXAMPLE 5

### Inhibition of the Growth of Human Hepatocellular Carcinoma HepG2 Cells by CKBM

The anti-tumor effect of CKBM was studied in HepG2 cell-bearing nude mice model. HepG2 cells were inoculated into nude mice and solid tumors were developed seven days after the inoculation. These nude mice were fed by intragastric injection with either the CKBM solution prepared from Example 1 at 0.4 ml/mouse and 0.8 ml/mouse, or water as controls every day. The treatment lasted for 14 consecutive days. Tumor sizes were measured by an electronic caliper before and after the treatment. The results show that the tumor growth was suppressed by the treatment of CKBM at 0.4 ml/mouse and 0.8 ml/mouse for 14 days. Among the two dosages, 0.8 ml/mouse was more effective than 0.4 ml/mouse.

The activities of two heart specific plasma enzymes (i.e., CK and LDH) in HepG2 cells-bearing mice were measured by methods known in the art to determine whether CKBM could cause heart failure, a common side effect of anti-hepatoma drugs such as doxorubicin. The results show that CKBM-treated mice exhibited similar CK and LDH activities to those in the control group. Furthermore, the toxicity of CKBM to the liver of HepG2 cells-bearing mice was also examined by measuring plasma AST and ALT activities. The results show that there was no significant change as to the activities of these two enzymes before and after CKBM treatment. Taken together, these results indicate that CKBM had no toxicity on the heart and liver of HepG2 cell-bearing mice.

### EXAMPLE 6

### Inhibition of the Growth of Human Gastric Cancer by CKBM

A subcutaneous tumor implantation model was used to investigate the effect of CKBM on the growth of human gastric cancer. Specifically, human gastric cancer tissues having a volume of about 1.5 mm³ were removed from a patient and implanted subcutaneously into the right dorsal of female athymic balb/c nude mice. Ten days after implantation, the mice are randomized into four treatment groups: a control group (0.8 ml water/mouse) and groups treated with the CKBM solution prepared from Example 1 at three different doses (0.2 ml, 0.4 ml, or 0.8 ml CKBM/mouse). Each mouse was fed intragastrically with either water or different dosage of CKBM daily for 14 or 28 days.

The effect of CKBM on tumor growth was first determined by examining tumor volume every 7 days after CKBM treatment. Tumor areas were measured by a caliper and calculated by the following formula: tumor volume (mm²)=d²×D/2, wherein d and D were the smallest and largest diameters, respectively. All the mice in the control group and the CKBM-treated group developed subcutaneous tumor after gastric cancer tissue implantation. Mice in the control group have tumors growing steadily during the 14- and 28-day experimental periods. CKBM-treated mice, however, showed a significant decrease in tumor growth starting from day 7. On day 21 and day 28, the tumor volumes in mice treated with CKBM at the dosages of 0.4 ml/mouse and 0.8 ml/mouse exhibited a 50% reduction when compared with the tumor volumes in mice of the control group. The inhibition of tumor growth was dose-dependent. CKBM did not affect the body weight of these mice during the whole experimental period.

In addition, the effect of CKBM on tumor growth was examined by measuring cancer cell proliferation after CKBM treatment. Gastric tumor tissue samples were obtained 14 or 28 days after CKBM treatment and subjected to histochemical staining of proliferating cell nuclear antigen (PCNA), an indicator of cell proliferation. Specifically, paraffin-embedded tumor samples that had been fixed in formalin were cut into sections of 5 μm in length. Sections were incubated in citrate buffer (0.01 M, pH 6.0) at 80° C. for 15 minutes and followed by digestion using pepsin (0.005%) in HCl (0.01 N, pH 2.0). After being washed with PBS (0.01 M, pH 7.4), sections were incubated with normal serum for 60 minutes. The sections were then subjected to immuno-staining using antibodies against mouse PCNA by standard methods. The total number of proliferating cells in a total of ten fields (×400) across and perpendicular at the center of the tumor was counted under a microscope. The results of cell proliferation were expressed as the number of PCNA-positive cells per field.

CKBM showed an anti-proliferation effect in mice containing gastric tumor tissues. For example, mice treated with CKBM for 14 days at a dosage of 0.8 ml/mouse showed a 30% reduction of cell proliferation compared with mice of the control group. Similarly, prolonged treatment of CKBM for 28 days inhibited the number of proliferating cells in gastric tumor in a dose-dependent manner.

Further, terminal deoxynucleotidyltransferase-mediated dUTP nick end-labeling method was used to determine cell apoptosis of tumor samples in paraffin-embedded tissue sections. The results show that CKBM treatment for 14 days did not result in anti-apoptotic effect in gastric cancer cells. Prolonged treatment of CKBM to 28 days, however, exhibited a dose-dependent induction of apoptosis in gastric cancer cells. At doses of 0.4 and 0.8 ml/mouse of CKBM, apoptosis was significantly increased by 76% and 97%, respectively, when compared with the corresponding control group. These results indicate that CKBM inhibited tumor growth by inducing cancer cell apoptosis.

### EXAMPLE 7

### Inhibition of the Growth of Prostate PC3 Cancer Cells by CKBM

Male athymic balb/c nude mice, aged between 6-8 weeks, were reared in IVC cages with isolated ventilation and free access to sterile food and drinking water. On the day of experiment, they were anesthetized by injecting i.p. xylazine (1.67 mg/ml) in an aqueous ketamine (1.67%) solution. After harvest and subculture in a medium, 5×105 prostate PC3 cells were implanted subcutaneously into the right dorsal of the mice. Subcutaneous nodules appeared on days 7-10 after tumor implantation. CKBM treatment (0.2, 0.4, or 0.8 ml/mouse of the CKBM solution prepared from Example 1) was given once daily by intragastric feeding and continued for 28 days. Mice in a control group received 0.8 ml of the vehicle. Tumor sizes were measured by their width and length with a caliper once in every several days from the 10th day after tumor implantation.

The results show that the tumor volumes in mice treated with CKBM at the dosages of 0.2 ml/mouse and 0.4 ml/mouse exhibited a 50% reduction on day 28 when compared with the tumor volumes in mice of the control group. Further, the results show that the tumor volumes in mice treated with CKBM at the dosage of 0.8 ml/mouse reduced by 66% on day 28, compared with the control group.

### EXAMPLE 8

### Inhibition of the Growth of Breast MDA-MB-231 Cancer Cells by CKBM

The inhibition of CKBM on the growth of breast MDA-MB-231 cancer cells was studied in a manner similar to that described in Example 7.

The results show that the tumor volumes in mice treated with the CKBM solution prepared from Example 1 at the dosages of 0.4 ml/mouse and 0.8 ml/mouse reduced by 40% and 50%, respectively, on day 28, compared with the control group.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A composition comprising the following four herbal extracts:
a Fructus Crataegi extract;
a Semen Zizyphus extract;
a Fructus Schizandrae extract; and
a Panax Ginseng extract.

2. The composition of claim 1, wherein the four herbal extracts are prepared from 3-30 g Fructus Crataegi, 3-30 g Semen Zizyphus, 4-20 g Fructus Schizandrae, and 1-15 g Panax ginseng; or prepared from Fructus Crataegi, Semen Zizyphus, Fructus Schizandrae, and Panax ginseng in quantities of the same ratio.

3. The composition of claim 1, further comprising an extract obtained from soybean, green bean, apple, yeast, or honey.

4. The composition of claim 1, wherein the composition is in a solution form containing an aqueous solvent or in a powder form.

5. The composition of claim 2, wherein the composition further comprises an extract obtained from yeast.

6. A food product comprising the composition of claim 1.

7. The food product of claim 6, wherein the food product is tea, soft drink, juice, milk, coffee, soup, beer, chewing gum, seasonings, cookies, cereals, and chocolates.

8. A pharmaceutical formulation comprising the composition of claim 1 and a pharmaceutically acceptable carrier.

9. The formulation of claim 8, wherein the formulation is a tablet, a capsule, powder, dispersion, a solution, or a gel.

10. A method of treating cancer, comprising administering to a subject in need thereof an effective amount of a composition according to claim 1.

11. The method of claim 10, wherein the cancer is liver, gastric, prostate, or breast cancer.

12. The method of claim 10, wherein the composition is according to claim 2.

13. The method of claim 10, wherein the composition is according to claim 3.

14. The method of claim 10, wherein the composition is administered orally.

15. A method of modulating an immune response in a subject, comprising administering to the subject in need thereof an effective amount of a composition according to claim 1.

16. The method of claim 15, wherein the immune response is modulated by induction of IL-6 or TNF-α.

17. The method of claim 15, wherein the composition is according to claim 2.

18. The method of claim 15, wherein the composition is according to claim 3.

19. The method of claim 15, wherein the composition is administered orally.

20. A method of increasing a cytokine in a cell, comprising contacting the cell with an effective amount of a composition according to claim 1.

21. The method of claim 20, wherein the induced cytokine is IL-6 or TNF-α.

22. The method of claim 20, wherein the cell is a lymphocyte.

23. The method of claim 22, wherein the lymphocyte is a peripheral blood mononuclear cell, a macrophage, or a T lymphocyte.

24. The method of claim 20, wherein the cell is presented in a cell culture, a tissue, or a subject.

25. The method of claim 20, wherein the composition is according to claim 2.

26. The method of claim 20, wherein the composition is according to claim 3.

27. A method of inducing an immune cell to release TFN-α, comprising contacting the immune cell with an effective amount of a composition according to claim 1.

28. The method of claim 27, wherein the immune cell is in an HIV carrying subject.

29. The method of claim 27, the composition is according claim 3.

30. A composition comprising at least two of the following four herbal extracts:
a Fructus Crataegi extract;
a Semen Zizyphus extract;
a Fructus Schizandrae extract; and
a Panax Ginseng extract.

31. The composition of claim 30, comprising at least three of the Fructus Crataegi extract, the Semen Zizyphus extract, the Fructus Schizandrae extract; and the Panax Ginseng extract.
